(19) United States
(12) Patent Application Publication
(10) Pub. No.: US 2025/0295636 A1
(43) Pub. Date: Sep. 25, 2025

(54) METHOD FOR PREPARING 4-[5-[BIS(2-CHLOROETHYL)AMINO]-1-METHYL-1H-BENZO[D]IMIDAZOL-2-YL]BUTYRIC ACID ALKYL ESTERS AND FORMYLATED DERIVATIVES

(71) Applicant: HERAEUS PRECIOUS METALS GMBH & CO. KG, Hanau (DE)

(72) Inventors: Tero KUNNARI, Hanau (DE); Steffen JOST, Hanau (DE); Holger RAUTER, Hanau (DE); Jenin GREIN, Hanau (DE)

(21) Appl. No.: 18/861,770

(22) PCT Filed: Jan. 16, 2023
(86) PCT No.: PCT/EP2023/050792

§ 371 Date: Oct. 30, 2024

(30) Foreign Application Priority Data
May 4, 2022 (EP) 22171496.7

(51) Int. Cl. A61K 31/4184; C07D 235/16

(52) U.S. Cl. CPC A61K 31/4184; C07D 235/16

(57) ABSTRACT

A method for preparing a 4-[5-[bis(2-chloroethyl)amino]-1-methyl-1 H-benzo[d]imidazol-2-yl]butyric acid alkyl ester, wherein 1 mol of 4-[5-[bis(2-hydroxyethyl)amino]-1-methyl-1H-benzo[d]imidazol-2-yl]butyric acid alkyl ester is reacted with 2.0 to 2.2 mol of chloromethylenedimethyliminium chloride or with >2.2 to 5 mol of chloromethylenedimethyliminium chloride to prepare a by-product in the form of a 4-[5-[bis(2-chloroethyl)amino]-4-formyl-1-methyl-1 H-benzo[d]imidazol-2-yl]butyric acid alkyl ester. The alkyl ester can be hydrolyzed to form 4-[5-[bis(2-chloroethyl)amino]-4-formyl-1-methyl-1H benzo[d]imidazol-2-yl]butyric acid that is usable in the field of medicine.

The invention relates to a method for preparing 4-[5-[bis(2-chloroethyl)amino]-1-methyl-1H-benzo[d]imidazol-2-yl]butyric acid alkyl esters and formylated derivatives thereof.

Ester hydrolysis can cause 4-[5-[bis(2-chloroethyl)amino]-1-methyl-1H-benzo[d]imidazol-2-yl]butyric acid alkyl esters to be converted to 4-[5-[bis(2-chloroethyl)amino]-1-methyl-1H-benzo[d]imidazol-2-yl]butyric acid. 4-[5-[bis(2-chloroethyl)amino]-1-methyl-1H-benzo[d]imidazol-2-yl]butyric acid is known as an anti-tumor agent going by the name “bendamustine.”

EP 2 468 716 A1 discloses a preparation method for 4-[5-[bis(2-chloroethyl)amino]-1-methyl-1H-benzo[d]imidazol-2-yl]butyric acid alkyl ester. The reactants used are 4-[5-[bis(2-hydroxyethyl)amino]-1-methyl-1H-benzo[d]imidazol-2-yl]butyric acid alkyl esters, the hydroxyethyl groups of which can be converted into chloroethyl groups by reaction with oxalyl chloride in the presence of dimethylformamide to form 4-[5-[bis(2-chloroethyl)amino]-1-methyl-1H-benzo[d]imidazol-2-yl]butyric acid alkyl esters. While work can be carried out stoichiometrically, i.e., with a molar ratio of 2.0 mol oxalyl chloride: 1 mol of 4-[5-[bis(2-hydroxyethyl)amino]-1-methyl-1H-benzo[d]imidazol-2-yl]butyric acid alkyl ester, EP 2 468 716 A1 recommends preferably using a stoichiometric excess corresponding to a molar ratio of ≥2.6 mol, in particular of at least 3.0 mol of oxalyl chloride per mol of 4-[5-[bis(2-hydroxyethyl)amino]-1-methyl-1H-benzo[d]imidazol-2-yl]butyric acid alkyl ester. The in-situ reaction of oxalyl chloride with dimethylformamide used here is difficult to control with regard to the completeness of the reaction or undesirable, interfering moisture in the reaction system. If the teachings of EP 2 468 716 A1 are followed and said superstoichiometric molar ratio recommended as preferred is used, the desired 4-[5-[bis(2-chloroethyl)amino]-1-methyl-1H-benzo[d]imidazol-2-yl]butyric acid alkyl ester in question is obtained in good yield, but with an undesirable yellow coloration, which is caused by an as yet unknown impurity and which is difficult to remove, even when treated with activated carbon. The intensity of the yellow coloration increases with the selected molar ratio of oxalyl chloride to 4-[5-[bis(2-hydroxyethyl)amino]-1-methyl-1H-benzo[d]imidazol-2-yl]butyric acid alkyl ester.

The object of the invention was to provide an improved preparation method for 4-[5-[bis(2-chloroethyl)amino]-1-methyl-1H-benzo[d]imidazol-2-yl]butyric acid alkyl esters starting from 4-[5-[bis(2-hydroxyethyl)amino]-1-methyl-1H-benzo[d]imidazol-2-yl]butyric acid alkyl esters.

The object can be achieved by a method for preparing a 4-[5-[bis(2-chloroethyl)amino]-1-methyl-1H-benzo[d]imidazol-2-yl]butyric acid alkyl ester by reacting a 4-[5-[bis(2-hydroxyethyl)amino]-1-methyl-1H-benzo[d]imidazol-2-yl]butyric acid alkyl ester with chloromethylenedimethyliminium chloride having the formula ClCH═N(CH₃)₂Cl.

In a first embodiment, the preparation method according to the invention can be carried out with a practically stoichiometric molar ratio in the range from 2.0 to 2.2 mol of chloromethylenedimethyliminium chloride per mol of 4-[5-[bis(2-hydroxyethyl)amino]-1-methyl-1H-benzo[d]imidazol-2-yl]butyric acid alkyl ester with a good yield of 4-[5-[bis(2-chloroethyl)amino]-1-methyl-1H-benzo[d]imidazol-2-yl]butyric acid alkyl ester and with little impurity and little by-product.

In a second embodiment, however, the preparation method according to the invention can also be carried out with a superstoichiometric molar ratio in the range from >2.2 to, for example, 5 mol of chloromethylenedimethyliminium chloride per mol of 4-[5-[bis(2-hydroxyethyl)amino]-1-methyl-1H-benzo[d]imidazol-2-yl]butyric acid alkyl ester. However, with a view to obtaining a high yield of 4-[5-[bis(2-chloroethyl)amino]-1-methyl-1H-benzo[d]imidazol-2-yl]butyric acid alkyl ester, the selection of a superstoichiometric molar ratio is less preferred.

Preferably, the 4-[5-[bis(2-hydroxyethyl)amino]-1-methyl-1H-benzo[d]imidazol-2-yl]butyric acid alkyl ester reacted with chloromethylenedimethyliminium chloride in the method according to the invention is a C₁₋₄ alkyl ester, in particular 4-[5-[bis(2-hydroxyethyl)amino]-1-methyl-1H-benzo[d]imidazol-2-yl]butyric acid ethyl ester; in other words, preferred embodiments of the method according to the invention consist in carrying it out using chloromethylenedimethyliminium chloride and a 4-[5-[bis(2-hydroxyethyl)amino]-1-methyl-1H-benzo[d]imidazol-2-yl]butyric acid C₁₋₄ alkyl ester, in particular 4-[5-[bis(2-hydroxyethyl)amino]-1-methyl-1H-benzo[d]imidazol-2-yl]butyric acid ethyl ester. Accordingly, a 4-[5-[bis(2-chloroethyl)amino]-1-methyl-1H-benzo[d]imidazol-2-yl]butyric acid C₁₋₄ alkyl ester, in particular the 4-[5-[bis(2-chloroethyl)amino]-1-methyl-1H-benzo[d]imidazol-2-yl]butyric acid ethyl ester, is obtained as the product of a preferred embodiment of the method according to the invention.

Chloromethylenedimethyliminium chloride is commercially available, for example from Sigma-Aldrich. It is a solid.

The synthesis method according to the invention can expediently be carried out in an aprotic, anhydrous (dried) organic solvent having an E_{T}(30) value in the range from 140 to 193 KJ/mol or in a mixture of such solvents. Examples of such solvents include chlorinated organic solvents, such as dichloromethane and chloroform, ethers, such as dioxane and tetrahydrofuran, and acetonitrile.

The two reactants used in the synthesis method according to the invention, the chloromethylenedimethyliminium chloride and a 4-[5-[bis(2-hydroxyethyl)amino]-1-methyl-1H-benzo[d]imidazol-2-yl]butyric acid alkyl ester, can react with one another in solution or in suspension, wherein in the latter case one or both reactants can be present in suspension. The two reactants can be added in any order, dissolved, suspended or as a solid.

In the method according to the invention, it is possible to work with a proportion, for example, in the range of 30 to 150 g reactant quantity (total quantity of both reactants) per liter of organic solvent (mixture).

It is useful to maintain a temperature in the reaction mixture, for example in the range from 0 to 60° C., both during the addition of the reactants and during the reaction.

For example, the reaction time can be in the range from 30 to 360 minutes. The reaction mixture is preferably mixed, in particular stirred.

After the reaction is complete, water can be added and a pH value in the range of 7 to 10, for example, can be adjusted by adding base. Examples of bases that can be used include ammonia, alkali hydroxide, alkali carbonate and alkali hydrogen carbonate.

If the reaction is carried out in a non-water-miscible solvent, the 4-[5-[bis(2-chloroethyl)amino]-1-methyl-1H-benzo[d]imidazol-2-yl]butyric acid alkyl ester formed in the method according to the invention is in the organic phase after addition of water and pH adjustment. Otherwise, the 4-[5-[bis(2-chloroethyl)amino]-1-methyl-1H-benzo[d]imidazol-2-yl]butyric acid alkyl ester formed can be converted into a non-water-miscible organic phase after addition of a non-water-miscible solvent, such as dichloromethane, and recovered and purified therefrom by conventional methods familiar to the organic chemist. Examples of such methods include crystallization methods, precipitation methods using an antisolvent and preparative chromatographic methods.

A 4-[5-[bis(2-chloroethyl)amino]-1-methyl-1H-benzo[d]imidazol-2-yl]butyric acid alkyl ester prepared according to the method of the invention can be subjected to ester hydrolysis to form 4-[5-[bis(2-chloroethyl)amino]-1-methyl-1H-benzo[d]imidazol-2-yl]butyric acid or its hydrochloride salt, for example analogous to the procedure known from EP 2 468 716 A1.

The preparation method according to the invention has a number of advantages over the preparation method known from EP 2 468 716 A1:
1. Using chloromethylenedimethyliminium chloride as a chlorinating reagent instead of oxalyl chloride/dimethylformamide is preferable from an occupational safety point of view in several respects. In addition to the advantages of avoiding the handling of the hazardous substances oxalyl chloride and dimethylformamide, the exothermic reaction and gas formation associated with the reaction of oxalyl chloride with dimethylformamide can be avoided.
2. Using chloromethylenedimethyliminium chloride allows an accurate dosage with regard to the adjustment of a desired stoichiometric or superstoichiometric molar ratio between chloromethylenedimethyliminium chloride and 4-[5-[bis(2-hydroxyethyl)amino]-1-methyl-1H-benzo[d]imidazol-2-yl]butyric acid alkyl ester; it also allows a subsequent dosage of chloromethylenedimethyliminium chloride if necessary.
3. Avoidability or reducibility of side reactions, in particular when carrying out the method according to the invention at a practically stoichiometric molar ratio in the range from 2.0 to 2.2 mol chloromethylenedimethyliminium chloride per mol of 4-[5-[bis(2-hydroxyethyl)amino]-1-methyl-1H-benzo[d]imidazol-2-yl]butyric acid alkyl ester.
4. Lower consumption of base when adjusting the pH value after completion of the reaction.

As mentioned above, the preparation method according to the invention in its second embodiment can be carried out with a superstoichiometric molar ratio in the range from >2.2 to, for example, 5 mol of chloromethylenedimethyliminium chloride per mol of 4-[5-[bis(2-hydroxyethyl)amino]-1-methyl-1H-benzo[d]imidazol-2-yl]butyric acid alkyl ester. When the method according to the invention is carried out in the second embodiment, for example with a molar ratio in the range from 2.6 to 5 mol of chloromethylenedimethyliminium chloride per mol of 4-[5-[bis(2-hydroxyethyl)amino]-1-methyl-1H-benzo[d]imidazol-2-yl]butyric acid C₁₋₄ alkyl ester, especially 4-[5-[bis(2-hydroxyethyl)amino]-1-methyl-1H-benzo[d]imidazol-2-yl]butyric acid ethyl ester, a yellow coloration analogous to the yellow coloration already mentioned at the beginning can be observed in the reaction system, which increases with increasing stoichiometric excess of chloromethylenedimethyliminium chloride. The substances causing the yellow coloration are method by-products of the method according to the invention in its second embodiment and have now been identified or isolated for the first time as the previously unknown and thus new compounds from the group of 4-[5-[bis(2-chloroethyl)amino]-4-formyl-1-methyl-1H-benzo[d]imidazol-2-yl]butyric acid C₁₋₄ alkyl esters. Apparently, when the method according to the invention is carried out in its second embodiment, a regioselective formylation of the actual main method product in position 4 of its benzo[d]imidazole backbone can occur. The corresponding previously unknown and thus novel 4-[5-[bis(2-chloroethyl)amino]-4-formyl-1-methyl-1H-benzo[d]imidazol-2-yl]butyric acid ethyl ester was obtained and characterized after separation from the main method product, the 4-[5-[bis(2-chloroethyl)amino]-1-methyl-1H-benzo[d]imidazol-2-yl]-butyric acid ethyl ester. Thus, 4-[5-[bis(2-chloroethyl)amino]-4-formyl-1-methyl-1H-benzo[d]imidazol-2-yl]butyric acid ethyl ester is a method by-product formed when carrying out the method according to the invention in its second embodiment. Due to its structural relationship with 4-[5-[bis(2-chloroethyl)amino]-1-methyl-1H-benzo[d]imidazol-2-yl]butyric acid, an anti-tumor effect can also be expected for 4-[5-[bis(2-chloroethyl)amino]-4-formyl-1-methyl-1H-benzo[d]imidazol-2-yl]butyric acid ethyl ester. Its aldehyde group as a reactive functionality can provide a starting point for manifold intra- and intermolecular derivatization reactions and thus for the preparation of anti-tumoral compounds, for example also for the formation of antibody conjugates.

The molar ratio selected when carrying out the method according to the invention in its second embodiment in the range from >2.2 mol to, for example, 5 mol of chloromethylenedimethyliminium chloride per mol of 4-[5-[bis(2-hydroxyethyl)amino]-1-methyl-1H-benzo[d]imidazol-2-yl]butyric acid alkyl ester, preferably 4-[5-[bis(2-hydroxyethyl)amino]-1-methyl-1H-benzo[d]imidazol-2-yl]butyric acid C₁₋₄ alkyl ester, especially 4-[5-[bis(2-hydroxyethyl)amino]-1-methyl-1H-benzo[d]imidazol-2-yl]butyric acid ethyl ester, can control the ratio between the main method product (4-[5-[bis(2-chloroethyl)amino]-1-methyl-1H-benzo[d]imidazol-2-yl]butyric acid alkyl ester, preferably 4-[5-[bis(2-chloroethyl)amino]-1-methyl-1H-benzo[d]imidazol-2-yl]butyric acid C1-4-alkyl ester, especially 4-[5-[bis(2-chloroethyl)amino]-1-methyl-1H-benzo[d]imidazol-2-yl]butyric acid ethyl ester) and the method by-product (4-[5-[bis(2-chloroethyl)amino]-4-formyl-1-methyl-1H-benzo[d]imidazol-2-yl]butyric acid alkyl ester, preferably 4-[5-[bis(2-chloroethyl)amino]-4-formyl-1-methyl-1H-benzo[d]imidazol-2-yl]butyric acid C1-4-alkyl ester, especially 4-[5-[bis(2-chloroethyl)amino]-4-formyl-1-methyl-1H-benzo[d]imidazol-2-yl]butyric acid ethyl ester). While practically no by-product is obtained with the method according to the first embodiment of the invention, a mixture of main method product and method by-product can be obtained with a molar ratio in the range from >2.2 to, for example, 5. As the molar ratio increases, the relative proportion of method by-product rises to around 5 mol % in addition to around 95 mol % of the main method product. An increase in the molar ratio to >5 mol of chloromethylenedimethyliminium chloride per mol of 4-[5-[bis(2-hydroxyethyl)amino]-1-methyl-1H-benzo[d]imidazol-2-yl]butyric acid alkyl ester is not recommended in the method according to invention in its second embodiment.

A 4-[5-[bis(2-chloroethyl)amino]-4-formyl-1-methyl-1H-benzo[d]imidazol-2-yl]butyric acid alkyl ester obtainable as a method by-product of the method according to the invention carried out in its second embodiment can be subjected to a further method step in the form of ester hydrolysis to form 4-[5-[bis(2-chloroethyl)amino]-4-formyl-1-methyl-1H-benzo[d]imidazol-2-yl]butyric acid, for example according to the procedure known from EP 2 468 716 A1, to which explicit reference is hereby made.

It is also possible to carry out the method according to the second embodiment of the invention and to subject the method by-product to ester hydrolysis together with the main method product. In other words, in this case, the product mixture consisting of said main method product and method by-product is not initially separated but subjected jointly to ester hydrolysis, likewise based, for example, on the procedure known from EP 2 468 716 A1, to which explicit reference is hereby made. The resulting mixture of 4-[5-[bis(2-chloroethyl)amino]-1-methyl-1H-benzo[d]imidazol-2-yl]butyric acid and 4-[5-[bis(2-chloroethyl)amino]-4-formyl-1-methyl-1H-benzo[d]imidazol-2-yl]butyric acid can then be separated using methods familiar to organic chemists, such as crystallization and/or preparative chromatographic methods.

As mentioned above, the ester hydrolysis mentioned several times herein can be carried out in accordance with the procedure known from EP 2 468 716 A1. For this purpose, a relevant isolated 4-[5-[bis(2-chloroethyl)amino]-1-methyl-1H-benzo[d]imidazol-2-yl]butyric acid alkyl ester, i.e., a corresponding formylated or non-formylated butyric acid alkyl ester, or a non-separated mixture of both, is preferably mixed with an acid. The acid used is preferably an inorganic acid, in particular hydrochloric acid. The acid is usually used as a concentrated acid, for example as concentrated hydrochloric acid. The acid can be added to the mixture of formylated and non-formylated ester by combining the acid with the ester (mixture) or with a solution thereof in a suitable organic solvent. The resulting mixture is then stirred, preferably at a temperature in the range from 10 to 80° C., more preferably at a temperature in the range from 15 to 70° C. and even more preferably at a temperature in the range from 20 to 60° C. The reaction time is preferably 30 minutes to six hours, more preferably one hour to four hours and even more preferably one hour to three hours. After the reaction, any organic components contained in the mixture, such as solvents or solvent residues, can be removed. This can preferably be done by distilling these organic components. The acid contained in the mixture is then removed from the hydrolyzed ester(s) by conventional means, preferably by distillation. The substituted butyric acid in question remains as the residue, i.e., the formylated substituted butyric acid (4-[5-[bis(2-chloroethyl)amino]-4-formyl-1-methyl-1H-benzo[d]imidazol-2-yl]butyric acid) or the non-formylated substituted butyric acid (4-[5-[bis(2-chloroethyl)amino]-1-methyl-1H-benzo[d]imidazol-2-yl]butyric acid) or the relevant mixture of formylated substituted butyric acid and non-formylated substituted butyric acid.

4-[5-[bis(2-chloroethyl)amino]-4-formyl-1-methyl-1H-benzo[d]imidazol-2-yl]butyric acid demonstrates an anti-tumoral effect, for example against the same types of cancer as 4-[5-[bis(2-chloroethyl)amino]-1-methyl-1H-benzo[d]imidazol-2-yl]butyric acid. This was demonstrated in experiments as shown in example 3 below. 4-[5-[bis(2-chloroethyl)amino]-4-formyl-1-methyl-1H-benzo[d]imidazol-2-yl]butyric acid and its pharmaceutically acceptable salts, for example a chloride, phosphate, sulphate, acetate, maleate, citrate or mesylate, even in the low IC50 concentration range of one hundred micromol per liter, demonstrate a cancer-cell-killing or cancer-cell-growth-inhibiting effect, for example on skin cancer, kidney cancer, lung cancer, brain tumors and pancreatic cancer. IC50 stands for “inhibition concentration,” at which 50% of the cancer cells are killed.

The aldehyde group as well as the carboxyl group of 4-[5-[bis(2-chloroethyl)amino]-4-formyl-1-methyl-1H-benzo[d]imidazol-2-yl]butyric acid as reactive functionalities can provide starting points for manifold intra- and intermolecular derivatization reactions and thus for the preparation of anti-tumorally active compounds, for example also for the formation of antibody conjugates.

4-[5-[bis(2-chloroethyl)amino]-4-formyl-1-methyl-1H-benzo[d]imidazol-2-yl]butyric acid, more precisely 4-[5-[bis(2-chloroethyl)amino]-4-formyl-1-methyl-1H-benzo[d]imidazol-2-yl]butyric acid in its function as active ingredient, and its pharmaceutically acceptable salts can accordingly be used as listed below:
1. in medicine, in particular in human medicine, specifically cancer medicine;
2. in or for the therapeutic treatment of cancer, in particular skin cancer, kidney cancer, lung cancer, brain tumors and pancreatic cancer;
3. as a drug, optionally in combination with other drugs, in particular as a drug in or for the therapeutic treatment of cancer, in particular skin cancer, kidney cancer, lung cancer, brain tumors and pancreatic cancer;
4. as a medicinal product, in particular as a medicinal product in or for the therapeutic treatment of cancer, in particular skin cancer, kidney cancer, lung cancer, brain tumors and pancreatic cancer;
5. in or for the manufacture of a medicinal product, in particular a medicinal product for the therapeutic treatment of cancer, in particular skin cancer, kidney cancer, lung cancer, brain tumors and pancreatic cancer.

### Example 1 (Reaction of Chloromethylenedimethyliminium Chloride With 4-[5-[bis(2-hydroxyethyl)amino]-1-methyl-1H-benzo[d]imidazol-2-yl]butyric Acid Ethyl Ester in a Molar Ratio of 2.0:1)

3.5 g chloromethylenedimethyliminium chloride and 55.8 g dichloromethane were cooled to 5° C. in a flask with stirring. To this suspension, a 1° C. cold solution of 5.0 g 4-[5-[bis(2-hydroxyethyl)amino]-1-methyl-1H-benzo[d]imidazol-2-yl]butyric acid ethyl ester in 28.7 g dichloromethane was added in portions, then heated to 40° C. and stirred at reflux for 9 h. Cooling to 10° C. then took place and 10 mL of water was added, a pH value between 8 and 10 was adjusted with 10 wt. % aqueous potassium carbonate solution, stirred for 10 min and the two-phase system was transferred to a separating funnel. The dichloromethane phase was separated. 46 mL of dichloromethane was added to the aqueous phase, shaken out and the dichloromethane phase separated. The combined dichloromethane phases were washed with 15 mL concentrated saline solution and the dichloromethane was removed under vacuum and by applying heat to the rotary evaporator. The residue was recrystallized from 10 mL ethyl acetate.

This yielded 4.92 g of 4-[5-[bis(2-chloroethyl)amino]-1-methyl-1H-benzo[d]imidazol-2-yl]butyric acid ethyl ester (=89% yield, based on the 5.0 g of 4-[5-[bis(2-hydroxyethyl)amino]-1-methyl-1H-benzo[d]imidazol-2-yl]butyric acid ethyl ester used).

Example 2 (Reaction of Chloromethylenedimethyliminium Chloride With 4-[5-[bis(2-hydroxyethyl)amino]-1-methyl-1H-benzo[d]imidazol-2-yl]butyric Acid Ethyl Ester in a Molar Ratio of 4.0:1, Followed by Ester Hydrolysis and Product Recovery)

7.0 g chloromethylenedimethyliminium chloride and 55.8 g dichloromethane were cooled to 5° C. in a flask with stirring. To this suspension, a 1° C. cold solution of 5.0 g 4-[5-[bis(2-hydroxyethyl)amino]-1-methyl-1H-benzo[d]imidazol-2-yl]butyric acid ethyl ester in 28.7 g dichloromethane was added in portions, then heated to 40° C. and stirred at reflux for 9 h. Cooling to 10° C. then took place and 10 ml of water was added, a pH value between 8 and 10 was adjusted with 10 wt. % aqueous potassium carbonate solution, stirred for 10 min and the two-phase system was transferred to a separating funnel. The dichloromethane phase was separated. 46 mL of dichloromethane was added to the aqueous phase, shaken out and the dichloromethane phase separated. The combined dichloromethane phases were washed with 15 mL of concentrated saline. The washed dichloromethane phase was then mixed with 5.78 g 32 wt. % hydrochloric acid and the dichloromethane was removed under vacuum and by applying heat to the rotary evaporator. A further 23.8 mL of 32 wt. % hydrochloric acid was added and stirring was continued for 90 min at an internal temperature of 40° C. After cooling to 20° C., 0.58 g activated carbon was added and it was stirred for 20 min, then the activated carbon was filtered off and rinsed with 24.8 mL water. Then, at a bath temperature of 45° C. and a vacuum of 15 mbar, the solution was concentrated to 16.0 g, a mixture of 19.75 g water and 2.87 g acetone was added and cooled to 5° C. within 60 min. The resulting suspension was stirred for 90 min at 5° C. internal temperature, filtered and washed with 23 mL cold water and 18 mL ethyl acetate. After drying in a vacuum, 3.58 g of 4-[5-[bis(2-chloroethyl)amino]-1-methyl-1H-benzo[d]imidazol-2-yl]butyric acid was obtained as the main product (=70% yield based on the 5.0 g of 4-[5-[bis(2-hydroxyethyl)amino]-1-methyl-1H-benzo[d]imidazol-2-yl]butyric acid ethyl ester used).

The acetone-based/water-based mother liquor obtained during filtration was freed of acetone under vacuum and a pH value between 3 and 4 was adjusted using 0.1M NaOH. The resulting weakly acidic solution was treated by reversed-phase chromatography (stationary phase: Dupont™ AmberChrom™ CG161M Chromatography Resin). A 0.1 wt. % aqueous trifluoroacetic acid solution with an acetonitrile gradient increasing from 0 to 50 wt. % was used as the eluent. The eluate was freed from the organic solvent in a vacuum and then dried by lyophilization. Ethyl acetate was added to the lyophilized material which recrystallized therefrom.

0.25 g 4-[5-[bis(2-chloroethyl)amino]-4-formyl-1-methyl-1H-benzo[d]imidazol-2-yl]butyric acid (=4.5% yield based on the 5.0 g 4-[5-[bis(2-hydroxyethyl)amino]-1-methyl-1H-benzo[d]imidazol-2-yl]butyric acid ethyl ester used) was yielded as a by-product in the form of a yellow solid.

The yellow solid was identified as 4-[5-[bis(2-chloroethyl)amino]-4-formyl-1-methyl-1H-benzo[d]imidazol-2-yl]butyric acid by UV-VIS, HRMS, ¹H-NMR and ¹³C-NMR spectroscopy.

UV-VIS in acetonitrile/water with 0.1% HCOOH (nm): 249 (peak, broad), 295 (shoulder), 405 (peak, broad).
HRMS: C₁₇H₂₁O₃N₃Cl₂, 386.10298 (delta m=−0.76 ppm)

### NMR Data (in CDCl₃)

| Position in the ring system | ¹H-NMR (ppm, intensity, | |
|---|---|---|
| or molecular fragment | multiplicity) | ¹³C-NMR (ppm) |
| 2 | — | 156.2 |
| 3a | — | 130.3 |
| 4 | — | 118.9 |
| 5 | — | 151.7 |
| 6-CH | 7.51, 1H, doublet | 122.2 |
| 7-CH | 7.93, 1H, doublet | 118.7 |
| 7a | — | 130.6 |
| NCH₃ | 4.03, 3H, singlet | 31.3 |
| CH₂CH₂CH₂COOH | 3.30, 2H, triplet | 24.6 |
| CH₂CH₂CH₂COOH | 2.11, 2H, multiplet | 22.8 |
| CH₂CH₂CH₂COOH | 2.49, 2H, triplet | 32.8 |
| COOH | broad | 174.1 |
| Cl(CH₂CH₂)₂N | 3.70, 4H, triplet | 57.3 |
| Cl(CH₂CH₂)₂N | 3.60, 4H, triplet | 41.9 |
| —CHO | 10.7, 1H, singlet | 190.2 |

### Example 3

Selected cell lines were incubated for 72 hours at 37° C. with different concentrations of 4-[5-[bis(2-chloroethyl)amino]-4-formyl-1-methyl-1H-benzo[d]imidazol-2-yl]butyric acid (concentrations ranging from 1.5 μmol to 5 mmol per liter). The cells were then incubated with MTS solution (Promega) for 1 hour, and the effects of 4-[5-[bis(2-chloroethyl)amino]-4-formyl-1-methyl-1H-benzo[d]imidazol-2-yl]butyric acid on cell proliferation were determined colorimetrically at 490 nm. Cell growth was expressed as a percentage of the corresponding control (0.5 wt. % dimethyl sulfoxide in water). The respective IC50 values were calculated on the basis of this data.

| Organ | Cell line | IC50 (mol per liter) |
|---|---|---|
| Skin | A375 | 1.3 · 10⁻³ |
| Kidneys | Caki-1 | 1.6 · 10⁻³ |
| Lung | Calu-6 | 1.3 · 10⁻³ |
| Brain | U87MG | 2.9 · 10⁻³ |
| Pancreas | BxPC-3 | 7.4 · 10⁻⁴ |

1. A method for preparing a 4-[5-[bis(2-chloroethyl)amino]-1-methyl-1H-benzo[d]imidazol-2-yl]butyric acid alkyl ester, wherein a 4-[5-[bis(2-hydroxyethyl)amino]-1-methyl-1H-benzo[d]imidazol-2-yl]butyric acid alkyl ester is reacted with chloromethylenedimethyliminium chloride.

2. The method according to claim 1, wherein the 4-[5-[bis(2-hydroxyethyl)amino]-1-methyl-1H-benzo[d]imidazol-2-yl]butyric acid alkyl ester is selected from C₁₋₄alkyl esters.

3. The method according to claim 1, wherein the 4-[5-[bis(2-hydroxyethyl)amino]-1-methyl-1H-benzo[d]imidazol-2-yl]butyric acid ethyl ester is used.

4. The method according to claim 1, wherein a molar ratio in the range from 2.0 to 2.2 mol of chloromethylenedimethyliminium chloride per mol of 4-[5-[bis(2-hydroxyethyl)amino]-1-methyl-1H-benzo[d]imidazol-2-yl]butyric acid alkyl ester is used.

5. The method according to claim 1, wherein a molar ratio in the range from >2.2 to 5 mol of chloromethylenedimethyliminium chloride per mol of 4-[5-[bis(2-hydroxyethyl)amino]-1-methyl-1H-benzo[d]imidazol-2-yl]butyric acid alkyl ester is used.

6. A 4-[5-[bis(2-chloroethyl)amino]-4-formyl-1-methyl-1H-benzo[d]imidazol-2-yl]butyric acid alkyl ester obtainable as a by-product of the method according to claim 5.

7. The method according to claim 2, wherein a molar ratio in the range from >2.2 to 5 mol of chloromethylenedimethyliminium chloride per mol of 4-[5-[bis(2-hydroxyethyl)amino]-1-methyl-1H-benzo[d]imidazol-2-yl]butyric acid C₁₋₄ alkyl ester is used.

8. A 4-[5-[bis(2-chloroethyl)amino]-4-formyl-1-methyl-1H-benzo[d]imidazol-2-yl]butyric acid C₁₋₄ alkyl ester obtainable as a by-product of the method according to claim 7.

9. The method according to claim 3, wherein a molar ratio in the range from >2.2 to 5 mol of chloromethylenedimethyliminium chloride per mol of 4-[5-[bis(2-hydroxyethyl)amino]-1-methyl-1H-benzo[d]imidazol-2-yl]butyric acid ethyl ester is used.

10. A 4-[5-[bis(2-chloroethyl)amino]-4-formyl-1-methyl-1H-benzo[d]imidazol-2-yl]butyric acid ethyl ester obtainable as a by-product of the method according to claim 9.

11. The method according to claim 5, wherein the method by-product obtained thereby is subjected to a subsequent ester hydrolysis to form 4-[5-[bis(2-chloroethyl)amino]-4-formyl-1-methyl-1H-benzo[d]imidazol-2-yl]butyric acid.

12. The method according to claim 11, wherein the method by-product is subjected to ester hydrolysis together with the main method product.

13. A 4-[5-[bis(2-chloroethyl)amino]-4-formyl-1-methyl-1H-benzo[d]imidazol-2-yl]butyric acid obtainable as the product of the method according to claim 11.

14. A 4-[5-[bis(2-chloroethyl)amino]-4-formyl-1-methyl-1H-benzo[d]imidazol-2-yl]butyric acid or a pharmaceutically acceptable salt thereof for use in medicine.
